(19) United States
(12) Patent Application Publication
(10) Pub. No.: US 2007/0066521 A1
(43) Pub. Date: Mar. 22, 2007

(54) Short RNA-binding proteins

(76) Inventor: Claude M. Fauquet, St Louis, MO (US)

Correspondence Address: BRYAN CAVE LLP, 211 NORTH BROADWAY, SUITE 3600, ST. LOUIS, MO, 63102-2750, US

(21) Appl. No.: 11/403,489

(22) Filed: Apr. 13, 2006

Related U.S. Application Data
(60) Provisional application No. 60/670,793, filed on Apr. 13, 2005.

(51) Int. Cl. A61K 48/00; A61K 38/56

(52) U.S. Cl. 514/12; 514/44

(57) ABSTRACT

The present invention provides compositions, kits, and methods that may be used to bind short RNA molecules, including miRNA and/or siRNA molecules. Such compositions and methods are useful in research and experimental systems to investigate the effects of, for example, miRNAs and/or siRNAs on endogenous and/or exogenous gene expression. Isolated polynucleotides and expression cassettes for producing the short RNA-binding compositions of the present invention are also provided.

### CROSS-REFERENCE TO RELATED APPLICATIONS

This application claims the benefit of U.S. provisional patent application Ser. No. 60/670,793, filed Apr. 13, 2005, which is incorporated herein by reference.

### FIELD OF THE INVENTION

The present invention relates to the field of molecular biology. More particularly, the invention relates to the field of short RNA molecule binding compositions, as well as methods of using such short RNA molecule binding compositions.

### BACKGROUND OF THE INVENTION

MicroRNAs (miRNAs) and short interfering RNAs (siRNAs) are endogenous small RNA molecules of approximately 21 nucleotides in length, which have been shown to regulate cellular development in plants and animals by selectively repressing gene expression. In addition, some miRNA and siRNA molecules have been shown to play critical roles in cell differentiation, including differentiation into malignant cells, in tissue development, and in virus function. Furthermore, miRNA and siRNA molecules have been shown to protect plants from viruses. Many life science investigators predict that miRNAs, in particular, represent a new aspect of universal gene regulation and, therefore, have been the subject of significant interest and research.

In light of the foregoing, it is desired to provide compositions and methods, which may be used to selectively bind, isolate and purify short, single-stranded RNA molecules, such as miRNAs, for use in research and experimental systems. More particularly, it is desired to provide compositions and methods, which may be used to selectively bind short, single-stranded RNA molecules to counteract the effects of, for example, miRNAs on gene expression in a system. In addition, it is desired to provide compositions and methods, which may be used to selectively bind short, double- and single-stranded RNA molecules to counteract the effects of, for example, siRNAs and miRNAs on gene expression in a system. It is also preferred that the compositions employed in such methods (i) are readily obtainable, (ii) are capable of binding to a solid substrate, and (iii) may be tethered (or otherwise attached or coupled to) other peptides or molecules without a significant loss of miRNA- and/or siRNA-binding activity.

### SUMMARY OF THE INVENTION

While siRNA and miRNA molecules have been shown to provide plants with some level of protection from viral infection, viruses have evolved to express certain proteins that interfere with the ability of such molecules to silence viral gene expression. In fact, the present invention exploits this natural defense mechanism of plant viruses, and provides compositions that may be used to selectively bind, isolate and/or purify short, single-stranded RNA molecules (such as miRNAs) and/or short, double-stranded RNA molecules (such as siRNAs).

One embodiment of the present invention relates to a method for selectively binding short, single-stranded RNA molecules, which comprises providing to a system an effective amount of a protein, wherein the protein comprises an amino acid sequence at least substantially similar to SEQ ID NO:1 (e.g., the A-AC4 protein, as defined herein). In such embodiments, the system may be a plant cell, including plant protoplasts. Additionally, in such embodiments, the system may consist of mammalian cells, insect cells, bacteria, fungi, yeast, and/or associated viruses. The invention provides that the short, single-stranded RNA-binding protein may be optionally coupled to at least one reporter or fusion molecule and/or immobilized to a solid or semi-solid substrate. In certain preferred embodiments, the protein is an A-AC4 protein, as defined herein.

Another embodiment of the present invention relates to a method for selectively binding short, single- and double-stranded RNA molecules, which comprises providing to a system an effective amount of a protein, wherein the protein comprises an amino acid sequence at least substantially similar to SEQ ID NO:2 (e.g., the S-AC4 protein, as defined herein). In such embodiments, the system may be a plant cell, including plant protoplasts. Additionally, in such embodiments, the system may consist of mammalian cells, insect cells, bacteria, fungi, yeast, and/or associated viruses. The invention provides that the short, single- and double-stranded RNA-binding protein may be optionally coupled to at least one reporter or fusion molecule and/or immobilized to a solid or semi-solid substrate. In certain preferred embodiments, the protein is an S-AC4 protein, as defined herein.

In certain embodiments of the invention, one or more of the short RNA-binding compositions described herein is provided directly to a system. For example, the invention provides that substantially isolated, purified and/or concentrated forms of such proteins, e.g., A-AC4, S-AC4, or fusions thereof, may be provided to a system. In other embodiments, the short RNA molecule binding compositions may be produced within the system. For example, the invention provides that a system may be provided with an expression cassette which comprises (i) a promoter sequence, (ii) a nucleic acid sequence encoding at least one short RNA-binding protein described herein, such as A-AC4 (SEQ ID NO:1) and/or S-AC4 (SEQ ID NO:2), and (iii) a termination sequence. In such embodiments, the system selected may be a plant cell, mammalian cell, insect cell, bacteria, fungi, yeast, or associated virus.

The methods of the present invention may further comprise a detection or monitoring step. More specifically, the invention provides that the concentration of short RNA molecules within a system may, optionally, be detected or monitored following the introduction of at least one short RNA-binding composition described herein.

According to still further embodiments of the present invention, kits are provided which may be used for binding short RNA molecules. In certain embodiments, for example, such kits comprise an isolated, purified and/or concentrated (i) single-stranded short RNA-binding proteins (such as a protein comprising the amino acid of SEQ ID NO:1) and/or (ii) single- and double-stranded short RNA-binding proteins (such as a protein comprising the amino acid of SEQ ID NO:2). In addition, in other related embodiments, the kits may comprise an expression cassettes and/or isolated polynucleotides which includes a nucleic acid sequence encoding a single-stranded (or single- and double-stranded) short RNA-binding protein described herein.

According to further embodiments, isolated short RNA-binding proteins are provided which, optionally, comprise appropriate buffers, stabilizers, solutions, additives, reagents, or solvents. In addition, the invention provides isolated polynucleotides and/or expression cassettes, which encode one or more of the short RNA-binding proteins described herein. Still further, the invention encompasses cell lines expressing one or more of the short RNA-binding proteins described herein.

The above-mentioned and additional features of the present invention are further illustrated in the Detailed Description contained herein. All references disclosed herein, including U.S. patents, are hereby incorporated by reference in their entirety as if each was incorporated individually.

### BRIEF DESCRIPTION OF THE FIGURES

FIG. 1: Transgenic expression of A-AC4 in Arabidopsis thaliana. Vector-transformed control plant (first plant from left to right). A-AC4 transformed Arabidopsis (second plant from left to right). Levels of miR159 (top-left blot), miR159* (top-right blot), and MYB-mRNA (middle blot) in vector-transformed control (“WT”) and A-AC4 transgenic plants (“A-AC4”). A-AC4 mRNA expression in A-AC4-transgenic Arabidopsis (bottom blot). Ethidium bromide (“EtBr”) stained gels served as loading controls.

FIG. 2: Expression levels of miRNA (top blot) and their target mRNA in virus-infected plants (bottom blot). Low molecular mass RNAs (21 nucleotides) isolated from wild-type control plant (“WT”), and from cassava (Manihot esculenta) and Nicotiana benthamiana infected with ACMV alone (“ACMV”), EACMCV alone (“EACMV”), and ACMV plus EACMCV (“Both”) (top blot). As positive control, synthetic miR159 and miRJAW RNA-oligonucleotides were used as size markers—and are shown on the left side (top blot). Total RNA (10 μg) isolated from virus-infected plants was probed with MYB DNA (bottom blot). Ethidium bromide (“EtBr”) stained gels served as loading controls.

FIG. 3: (from left to right, gels 1, 2, and 3) Electrophoretic mobility shift assays using purified A-AC4 and E-AC2 proteins, and synthetic miR159, miR159*, miR-lin4, miR-lin4*, single-stranded sense (ss-siRNA) and antisense-strands (as-siRNA) of siGFP as indicated at the top of each blot. Lane 1=control (5′-labelled corresponding oligonucleotide); Lane 2=GST protein; Lane 3=GST::A-AC4 fusion protein; and Lane 4=GST::E-AC2 fusion protein. (from left to right, gel 4) High affinity binding of A-AC4 with miR159 in competition binding assay using a 10-, 50- and 100-fold excess of unlabelled miR159.

FIG. 4: (top-left) Northern blot analysis of miR159 in (a) the input samples (“Input”) and (b) remaining in the supernatant of extracts obtained from Arabidopsis leaf-derived control protoplasts (“Wild type”), and protoplasts transfected with 35S::GFP (“GFP”) or 35S::A-AC4-GFP (“A-AC4-GFP”), after incubation with 2′-O-methyl miR159 oligonucleotide (“miR159”) or 2′-O-methyl Pp-luc oligonucleotide (defined below) (“UR”). (lower-left) Western blot of fractionated extract from protoplasts transfected with 35S:GFP or A-AC4::GFP fusion construct (wherein the extract was incubated with 2′-O-methyl miR159* oligonucleotide (“miR159”) or 2′-O-methyl Pp-luc oligonucleotide (“UR”) prior to fractionation and blotting). (top-right) Northern blot analysis for presence of miR159 sequences following the immunoprecipitation example as described below (and presented in the lower panels (blots) of this Figure). (lower-right) Western blot analysis for presence of GFP protein following the immunoprecipitation example described below.

FIG. 5: Levels of miRNAs and ta-siRNAs, and their target mRNA accumulation, in transgenic Arabidopsis plants expressing S-AC4. In all of the electrophoretic gels and hybridization blots shown in FIG. 5, low molecular mass RNA (40 μg) isolated from young Arabidopsis tissues transformed with the S-AC4, S-AC2, or control (Con) vectors described herein was loaded in each lane. Panel (A) shows the accumulation levels of 8 miRNAs as indicated at the top of each blot. Ethidium bromide stained gels (EtBr) served as loading controls. Panel (B) shows levels of ta-siRNAs (TAS1, TAS2, and TAS3) in S-AC4, S-AC2 and vector-transformed control plants as indicated. For clarity purposes, contrasted versions of TAS1 and TAS2 blots were shown at the bottom of the respective blots. Ethidium bromide stained gels served as loading controls. Panel (C) refers to a β-elimination assay to determine the chemical nature of miRNAs and ta-siRNAs in S-AC4, S-AC2 and vector-transformed control plants (Con). Signs (+) and (−) represent chemically-treated and non-treated samples, respectively. Detection of miR159 (left) and ta-siR255 (right); samples subjected to β-elimination (+) or not (−). In the miR159 blot, as an internal control, a synthetic 12 nucleotide RNA (marked as miR159-12nt) was included to ensure completion of the reaction. Panel (D) Total RNA (20 μg) isolated from young leaf (“Leaf”) or influoresence tissue (“Inf”) obtained from S-AC4, S-AC2 and vector-transformed control plants (Con) was loaded in each blot. Blots were hybridized with gene specific probes as indicated at the top of each blot. Ethidium bromide stained gels served as loading controls.

FIG. 6: Electrophoretic mobility shift assays (EMSA) using purified S-AC4-GST fusion protein and synthetic miRNA sequences of plants, C. elegans and mammal sources.
Panel (A): EMSA of S-AC4 protein with miR159, miR159* and duplex miR159:miR159*—lane (1) represents short RNAs alone, lane (2) represents short RNAs and GST, and lane (3) represents short RNAs and S-AC4-GST fusion protein.
Panels (B, C, D and E): EMSAs of S-AC4 protein with various single- and double-stranded short RNA molecules. Lanes (1) and (2) represent short RNAs alone and short RNAs with S-AC4-GST fusion protein, respectively. (B) EMSA of S-AC4 protein, with miR165, miR165* and miR165::miR165*; (C) with 2′-O-methyl modified miR165, miR165* and miR165::miR165* at the last ribonucleotide; (D) with lin4, lin4* and lin4::lin4 duplex, a C. elegans miRNA sequence; and (E) with mammalian miR34, miR34* and miR34::miR34*.
Panel (F): Differential binding capacity of S-AC4 and A-AC4 GST fusion proteins with single-stranded and double-stranded (i.e., duplex) forms of miRNAs. Sequences of miR165, miR165* and miR165::miR165* duplex were used in the subject binding assays. Lane (1) represents short RNAs alone, lane (2) represents short RNAs with S-AC4-GST, and lane (3) represents short RNAs with A-AC4-GST.
Panels (G, H, I and J): EMSA of S-AC4-GST fusion protein with ta-siRNAs and siRNAs. In (G, H, I and J), lane (1) represents short RNAs alone and lane (2) represents short RNAs with S-AC4 protein. (G) Synthetic ta-siR255, ta-siR255* and a duplex form of ta-siR255 (ta-siR255-d) were used in this binding assay. (H) Synthetic siRNA designed to target a GFP sequence: sense strand (siGFP), anti-sense strand (as-siGFP), and double-stranded form of siGFP (siGFP-d) were used in this binding assay. (I) Absence of S-AC4 protein binding with long RNAs representing the precursor sequence of miR159. A single-stranded form of long RNA encompassing the miR159 sequence (ss-longRNA) and a duplex form of two single-strands comprising the entire length of pre-miR159 sequence (ds-longRNA) were used in this binding assay. (J) Absence of S-AC4 protein binding with synthetic sense (DNA-s), anti-sense (DNA-as), and duplex form (DNA-d) DNA sequence corresponding to miR165. In this binding assay, lanes (1) and (3) represent short RNAs alone and lanes (2) and (4) represent short RNAs with S-AC4 protein.

Panels (K and L): Competition binding assay of S-AC4 protein to single-stranded mi165 (K) and miR165-duplex (L) using an excess of unlabelled miR165 and miR165-duplex as shown under the triangle (10, 50 and 100-fold).

BRIEF DESCRIPTION OF THE SEQUENCE LISTING

| | |
|---|---|
| SEQ ID NO:1 | Amino acid sequence of A-AC4 |
| SEQ ID NO:2 | Amino acid sequence of S-AC4 |
| SEQ ID NO:3 | Non-limiting example of nucleic |
| | acid sequence encoding A-AC4 |
| SEQ ID NO:4 | Non-limiting example of nucleic |
| | acid sequence encoding S-AC4 |
| SEQ ID NO:5 | miR159 |
| SEQ ID NO:6 | Coding sequence of ACMV-AC2 |
| SEQ ID NO:7 | Coding sequence of EACMCV-AC2 |
| SEQ ID NO:8 | Coding sequence of EACMCV-AC4 |
| SEQ ID NO:9 | Coding sequence of MYB |
| SEQ ID NO:10 | ACMV-AC2 amino acid sequence |
| SEQ ID NO:11 | EACMCV-AC4 amino acid sequence |
| SEQ ID NO:12 | EACMCV-AC2 amino acid sequence |
| SEQ ID NO:13 | miR159* |
| SEQ ID NO:14 | Anti-sense strand of siRNA |
| | (siGFP) |
| SEQ ID NO:15 | miR-lin4 |
| SEQ ID NO:16 | miR-lin4* |
| SEQ ID NO:17 | Sense strand of siRNA (siGFP) |
| SEQ ID NO:18 | miR165 |
| SEQ ID NO:19 | miR166 |
| SEQ ID NO:20 | miR171 |
| SEQ ID NO:21 | miRJAW |
| SEQ ID NO:22 | Pp-luc sequence |
| SEQ ID NO:23 | Antisense siRNA strand targeting |
| | the firefly (Photinus pyralis) |
| | luciferase (Pp-luc) |
| SEQ ID NO:24 | miR160 |
| SEQ ID NO:25 | miR168 |
| SEQ ID NO:26 | miR173 |
| SEQ ID NO:27 | miR390 |
| SEQ ID NO:28 | miR396 |
| SEQ ID NO:29 | ta-siR255 (TAS 1) |
| SEQ ID NO:30 | ta-siR1511 (TAS 2) |
| SEQ ID NO:31 | ta-siR1769 (TAS 3) |
| SEQ ID NO:32 | ta-siR1778 (TAS 3) |
| SEQ ID NO:33 | miR165* |
| SEQ ID NO:34 | miR34 |
| SEQ ID NO:35 | miR34* |
| SEQ ID NO:36 | DmiR-165 |
| SEQ ID NO:37 | DmiR-165* |
| SEQ ID NO:38 | ta-siRNA siR255 |
| SEQ ID NO:39 | ta-siRNA siR255* |
| SEQ ID NO:40 | miR159-precursor (first-strand) |
| SEQ ID NO:41 | miR159-precursor (second-strand) |

### DETAILED DESCRIPTION OF THE INVENTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used, and made without departing from the scope and spirit of the invention.

The present invention relates generally to compositions, kits, and methods that may be used to bind short RNA molecules, including without limitation miRNA and/or siRNA molecules. Such compositions, kits, and methods are useful in research and experimental systems to investigate the effects of siRNAs and miRNAs on endogenous and/or exogenous gene expression and corresponding mRNA levels.

One embodiment of the present invention relates to a method for binding short RNA molecules which comprises providing to a system an effective amount of at least one short RNA-binding protein described herein, such as the A-AC4 and/or S-AC4 proteins (or sequences that are substantially similar to either of the foregoing). In such embodiments, the system may be a plant cell. Additionally, the system may consist of mammalian cells, insect cells, bacteria, fungi, yeast, and/or associated viruses. As used herein, the phrases “short RNA molecule,” “short RNA molecules,” “short RNAs,” and similar phrases, mean single-stranded and/or double-stranded ribonucleic acid (RNA) molecules which, preferably, range from 19 to 26 nucleotides in length, including without limitation microRNAs (“miRNAs”) and short interfering RNAs (“siRNAs”). In many cases, miRNAs are single-stranded short RNA molecules, whereas siRNAs are double-stranded short RNA molecules. The phrases “binding short RNA molecules,” “bind short RNA molecules,” “binding short RNAs,” “bind short RNAs” and similar phrases mean capturing and/or localizing short RNA molecules using one or more of the short RNA molecule binding proteins described herein. Similarly, the phrases “short RNA-binding proteins,” “short RNA molecule binding proteins,” and similar phrases refer to the proteins described herein which are capable of binding short RNAs, including without limitation A-AC4, S-AC4, combinations thereof, and sequences that are substantially similar to any of the foregoing.

In a preferred embodiment of the present invention, an isolated peptide derived from the African cassava mosaic virus (ACMV), namely, the ACMV-AC4 protein (referred to herein as “A-AC4”) (SEQ ID NO:1), is used to bind short RNA molecules as described herein. Still further, the invention provides that isolated proteins which are (a) identical to A-AC4 and derived from a source other than ACMV; (b) biologically equivalent to A-AC4; (c) conservatively modified variants of A-AC4; or (d) which comprise an amino acid sequence that is substantially similar to A-AC4, or fragments thereof, may be used to bind short RNAs, as described herein. In certain preferred embodiments, the A-AC4 protein is used to preferentially and selectively bind single-stranded, short RNA molecules, such as miRNAs.

In another preferred embodiment of the present invention, an isolated peptide derived from the Sri Lankan cassava mosaic virus (SLCMV), namely, the SLCMV-AC4 protein (referred to herein as “S-AC4”) (SEQ ID NO:2), is used to bind short RNA molecules as described herein. Still further, the invention provides that isolated proteins which are (a) identical to S-AC4 and derived from a source other than SLCMV; (b) biologically equivalent to S-AC4; (c) conservatively modified variants of S-AC4; or (d) which comprise an amino acid sequence that is substantially similar to S-AC4, or fragments thereof, may be used to bind short RNAs, as described herein. In certain preferred embodiments, the S-AC4 protein is used to preferentially and selectively bind single- and double-stranded, short RNA molecules, such as miRNAs and siRNAs.

Two amino acid sequences (i.e., peptides, polypeptides, proteins, etc.) are said to be “identical” if the sequence of amino acid residues in the two sequences is the same when aligned for maximum correspondence as described below—regardless of whether such proteins are derived from different sources. Thus, the term “identical,” in the context of two or more peptide sequences, refers to two or more sequences or subsequences that are the same (or have a specified percentage of amino acid residues that are the same), when compared and aligned for maximum correspondence over a comparison window, as measured using one of the sequence comparison algorithms described below (or by manual alignment and visual inspection).

With respect to the short RNA-binding proteins described herein, the term “isolated” means a protein or a polypeptide that has been separated from components that accompany it in its natural state. A protein is isolated, for example, when at least about 50 to 75% of a sample exhibits a single polypeptide sequence. An isolated protein will typically comprise about 60 to 90% w/w of a protein sample, more usually about 95%, and preferably will be over about 99% pure. Protein purity or homogeneity may be indicated by a number of means well known in the art, such as Sodium Dodecyl Sulfate—Polyacrylamide Gel Electrophoresis (SDS-PAGE) of a protein sample followed by visualizing a single polypeptide band upon staining the gel. For certain purposes, high pressure liquid chromatography (HPLC) or other means well known in the art may provide higher resolution for purification.

As used herein, the term “biologically equivalent” refers to a protein having structural, regulatory, or biochemical functions of a naturally occurring molecule, such as A-AC4 or S-AC4. More particularly, for example, a protein is “biologically equivalent” if it is capable of binding short RNA molecules in a similar manner as A-AC4 or S-AC4, as shown and described herein. Non-limiting examples of methods that may be used to assess whether a protein is biologically equivalent based on its ability to bind short RNA molecules are shown and described below.

The phrase “substantially similar,” in the context of two peptides, refers to sequences or subsequences that have at least 60%, preferably at least 70%, still more preferably at least 80%, and most preferably at least 90%, amino acid residue similarity when aligned for maximum correspondence over a comparison window (as measured using one of the sequence comparison algorithms set forth below or by manual alignment and visual inspection).

For sequence comparison to determine whether a first sequence is substantially similar to another, generally, one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence similarity for the test sequences relative to the reference sequence, based on the program parameters.

A “comparison window,” as used herein, includes reference to a segment of any one of the number of contiguous positions selected from the group consisting of from about 20 to 600, usually about 50 to about 200, more usually about 100 to about 150, in which a sequence may be compared to a reference sequence of the same number of contiguous positions after the two sequences are optimally aligned. Methods of alignment of sequences for comparison are well known in the art. Optimal alignment of sequences for comparison may be conducted, e.g., by the local homology algorithm of Smith & Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat., Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), or by manual alignment and visual inspection.

Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov). The BLAST algorithm also performs a statistical analysis of the similarity between two sequences (see, e.g., Karlin and Altschul, Proc. Nat. Acad. Sci. USA 90:5873-5787 (1993)). One measure of similarity provided by the BLAST algorithm is the smallest sum probability (P(N)), which provides an indication of the probability that a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

A further indication that two peptides are “substantially similar” is that a first polypeptide is immunologically cross-reactive with antibodies raised against a second polypeptide. In many cases, a polypeptide is substantially similar to a second polypeptide, for example, where the two polypeptides differ primarily by conservative substitutions.

The invention provides that the short RNA binding proteins of the present invention include “conservatively modified variants” of A-AC4 and S-AC4, wherein certain amino acid residues of the naturally occurring A-AC4 or S-AC4 protein are substituted with chemically similar residues. The following six groups each contain amino acids that are conservative substitutions for one another:

Alanine (A), Serine (S), Threonine (T);

Aspartic acid (D), Glutamic acid (E);

Asparagine (N), Glutamine (Q);

Arginine (R), Lysine (K);

Isoleucine (I), Leucine (L), Methionine (M), Valine (V); and

Phenylalanine (F), Tyrosine (Y), Tryptophan (W).

The invention further provides that the short RNA-binding proteins described herein may be optionally (directly or indirectly through a spacer arm) coupled to at least one reporter molecule, such as the green fluorescent protein (GFP). Additionally, the short RNA binding proteins of the current invention may be immobilized to a solid or semi-solid substrate (directly or indirectly through a spacer arm), such as polystyrene, polypropylene, agarose, cellulose, nitrocellulose, and other solid or semi-solid substrates which are commonly used throughout the research community.

In another embodiment of the present invention, methods are provided for producing one or more of the short RNA-binding proteins described herein. Such methods include providing an appropriate host, such as a plant cell, insect cell, mammalian cell, bacteria, yeast, or others, with an expression cassette of the present invention and obtaining the short RNA-binding protein therefrom. The expression cassettes of the present invention, preferably, contain an expression control sequence, such as a promoter, that is operably linked to a nucleic acid sequence that encodes at least one short RNA-binding protein, such as SEQ ID NO:3 (encoding A-AC4 (SEQ ID NO:1)) or SEQ ID NO:4 (encoding S-AC4 (SEQ ID NO:2)), and, preferably, a terminator sequence operably linked to the short RNA-binding protein-encoding sequence.

As used herein, the phrase “expression cassette” is a replicatable vehicle that carries, and is capable of mediating the expression of, a DNA sequence encoding the short RNA-binding proteins described herein. In the present context, the term “replicatable” means that the cassette is able to replicate in a given type of host, including a plant cell, insect cell, mammalian cell, bacteria, yeast, or others, into which it has been introduced. Immediately upstream of the nucleic acid sequence(s) encoding the short RNA-binding protein(s), there may be provided a sequence coding for a signal peptide, the presence of which ensures secretion of the encoded polypeptide expressed by host cells harboring the cassette. The signal sequence may be naturally associated with the selected nucleic acid sequence or of another origin.

The expression cassette may be any cassette that may conveniently be subjected to recombinant DNA procedures, and the choice of cassette will often depend on the host cell into which it is to be introduced. Thus, the cassette may be an autonomously replicating vector, i.e., a vector that exists as an extrachromosomal entity, the replication of which is independent of chromosomal replication. Examples of such a vector are a plasmid, phage, cosmid or mini-chromosome. Alternatively, the cassette may be one which, when introduced into a host cell, is integrated in the host cell genome and is replicated together with the chromosome(s) into which it has been integrated. Non-limiting examples of suitable cassettes are described further below. The expression cassettes of the present invention may carry any of the nucleic acid sequences, and be used for the expression of any of the short RNA-binding proteins, described herein.

An “expression control sequence,” as used herein, means an array of nucleic acid control sequences that direct transcription of an operably linked nucleic acid, such as a sequence encoding A-AC4, S-AC4, combinations thereof, or any other short RNA binding protein of the present invention. An example of such an expression control sequence is a “promoter,” including constitutive and inducible promoters. Promoters include necessary nucleic acid sequences near the start site of transcription. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under chemical, biochemical, environmental, or developmental regulation. In addition, promoters may be active in a tissue-specific manner. The term “operably linked” refers to a functional linkage between a nucleic acid expression control sequence (such as a promoter or an array of transcription factor binding sites) and a second nucleic acid sequence, wherein the expression control sequence directs transcription of the nucleic acid corresponding to the second sequence, e.g., an A-AC4 or S-AC4 encoding sequence or other short RNA-binding protein of the present invention.

The present specification provides non-limiting examples of nucleic acid sequences that may be used to express (or produce) representative short RNA-binding proteins of the present invention. For example, SEQ ID NO:3 is an example sequence that encodes A-AC4 (SEQ ID NO:1), whereas SEQ ID NO:4 is an example sequence that encodes S-AC4 (SEQ ID NO:2). Those of ordinary skill in the art will recognize, however, that because of codon degeneracy, a number of nucleic acid sequences will encode the same protein. Such conservatively modified variants are specifically within the scope of the present invention. In addition, the present invention specifically includes those nucleic acid sequences that (1) are identical (regardless of the source of origin) or substantially similar (determined as described below) to those described herein or (2) that encode short RNA-binding proteins that are biologically equivalent to, substantially similar to, or represent conservatively modified variants of, wild type short RNA-binding proteins, such as the naturally occurring A-AC4 or S-AC4 proteins (e.g., resulting from conservative substitutions of amino acids in the naturally occurring protein).

More specifically, a first nucleic acid sequence may be determined to be “substantially similar” to a second sequence (i.e., the “target”) if the first sequence (i.e., the “probe”) specifically hybridizes to the second sequence under stringent hybridization conditions. The phrase “specifically hybridizes to” refers to the binding, duplexing, or hybridizing of a molecule only to a particular nucleotide sequence under stringent hybridization conditions when that sequence is present in a complex mixture (e.g., total cellular or library DNA or RNA). The phrase “stringent hybridization conditions” refers to conditions under which a probe will hybridize to its target sequence, typically in a complex mixture of nucleic acid sequences, but to no other sequences.

Stringent conditions are sequence-dependent and will vary in different circumstances. Longer sequences of similar base composition hybridize specifically at higher temperatures. An extensive guide to the hybridization of nucleic acids is found in Tijssen, Techniques in Biochemistry and Molecular Biology—Hybridization with Nucleic Probes, “Overview of Principles of Hybridization and the Strategy of Nucleic Acid Assays” (1993). Generally, highly stringent conditions are selected to be about 5-10° C. lower than the thermal melting point (Tₘ) for the specific sequence at a defined ionic strength and pH. Low stringency conditions are generally selected to be about 15-30° C. below the Tₘ. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents, such as formamide. For selective or specific hybridization, a positive signal is at least two times background, preferably 10 times background hybridization.

Nucleic acids that do not hybridize to each other under stringent conditions are still “substantially similar” if the peptides that they encode are substantially similar to one another (as describe above). This occurs, for example, when a copy of a nucleic acid is created using the maximum codon degeneracy permitted by the genetic code.

As stated, the short RNA-binding proteins of the present invention may be produced using any of various means well-known in the art. For purposes of illustration, the short RNA-binding proteins may be expressed, via recombinant DNA technology, in host cells such as bacteria, yeast, plant, insect, and cultured mammalian cells. In certain embodiments, for example, the sequence encoding the desired short-RNA binding protein may be designed to express such peptide as a soluble or secreted molecule, wherein the soluble peptide may be recovered from the culture media. Purification from such expression systems may be accomplished using appropriate detergents, lipid micelles, and methods well known to those skilled in the art.

In addition, proteins with the same functionality as that of the present invention may be constructed de-novo, or by using the process of “directed evolution,” wherein a starting molecule- or protein-encoding gene (e.g., genes encoding A-AC4 or S-AC4) is mutagenized or modified in successive cycles of selection until the desired functionality is obtained. For example, an assay for use in directed evolution may comprise a targeted miRNA, a target reporter mRNA (such as mRNA encoding Luciferase), and the evolved gene-encoded protein. Luciferase activity would increase as successive cycles produce a gene encoding a functional miRNA-binding protein having enhanced or improved activity, relative to the reference A-AC4 or S-AC4 protein. Luciferase compositions and assays are commercially available (e.g., Promega Corporation, Madison, Wis.).

The expression systems that may be used for purposes of the invention include, without limitation, microorganisms such as (1) bacteria (e.g., E. coli, B. subtilis) transformed with recombinant bacteriophage DNA, plasmid DNA, or cosmid DNA vectors, which contain expression cassettes of the present invention (i.e., which contain sequences that encode at least one short RNA-binding protein described herein, such as SEQ ID NO:1, SEQ ID NO:2, or sequences that are substantially similar thereto); (2) yeast (e.g., Saccharomyces, Pichia) transformed with recombinant yeast expression vectors containing an expression cassette of the present invention; (3) insect cell systems infected with recombinant virus expression vectors (e.g., baculovirus) containing an expression cassette of the present invention; (4) plant cell systems infected with recombinant virus expression vectors (e.g., cauliflower mosaic virus, CaMV; tobacco mosaic virus, TMV; cowpea mosaic virus, CPMV; potato virus X PVX; and tobacco rattle virus, TRV) or transformed with recombinant plasmid expression vectors (e.g., the Ti plasmid) containing an expression cassette of the present invention; or (5) mammalian cell systems (e.g., COS, CHO, BHK, 293, 3T3) harboring recombinant expression constructs containing short RNA-binding protein-encoding sequences of the present invention operably linked to promoters derived from the genome of mammalian cells (e.g., metallothionein promoter) or from mammalian viruses (e.g., the adenovirus late promoter; the vaccinia virus 7.5K promoter; baculovirus system or others).

In bacterial systems, for example, a number of expression vectors may be employed. In certain embodiments, for example, vectors that direct the expression of high levels of fusion protein products (which are easily purified) may be desirable. Such vectors include, without limitation, the E. coli expression vector pUR278 (Ruther et al. (1983) EMBO J. 2:1791), in which a sequence encoding a short RNA-binding protein of the present invention, such as SEQ ID NO:3 (encoding A-AC4 (SEQ ID NO:1)) or SEQ ID NO:4 (encoding S-AC4 (SEQ ID NO:2)), may be ligated into the vector in frame with a lacZ coding region, wherein a fusion protein is produced; pIN vectors (Inouye and Inouye (1985) Nucleic Acids Res. 13:3101-3109; Van Heeke and Schuster (1989) J. Biol. Chem. 264:5503-5509); and the like.

pGEX vectors may also be used to express the short RNA-binding proteins of the present invention as fusion proteins with glutathione S-transferase (GST). In general, such fusion proteins are soluble and may be easily purified from lysed cells by adsorption to glutathione-agarose beads followed by elution in the presence of free glutathione. The pGEX vectors are, preferably, designed to include thrombin or factor Xa protease cleavage sites to allow the short RNA-binding protein to be released from the GST moiety. Importantly, as shown in the examples below, the activity of the short RNA-binding proteins described herein is not significantly impacted when expressed as a fusion protein. In the examples below, A-AC4 was expressed as a fusion protein with GFP, while maintaining its ability to bind short RNA molecules as shown and described.

In an insect system, for example, Autographa californica Nucleopolyhedrovirus (ACNPV) may be used as a vector to express short RNA-binding proteins. The virus may be cultured in Spodoptera frugiperda cells. A sequence encoding A-AC4 or S-AC4 (or substantially similar sequences), for example, may be cloned into non-essential regions (for example the polyhedrin gene) of the virus and placed under control of an AcNPV promoter (for example the polyhedrin promoter). Successful insertion of the short RNA-binding protein-encoding sequence will, preferably, inactivate the polyhedrin gene and production of non-occluded recombinant virus (i.e., virus lacking the proteinaceous coat—encoded by the polyhedrin gene). Such recombinant viruses may then be used to infect Spodoptera frugiperda cells in which the inserted nucleic acid sequence is expressed (See, e.g., Smith et al. (1983) J. Virol. 46: 584; and Smith, U.S. Pat. No. 4,215,051).

In mammalian host cells, for example, a number of viral-based expression systems may be utilized. For example, an adenovirus may be used as an expression vector, wherein a sequence encoding a short RNA-binding protein may be ligated to an adenovirus transcription/translation control complex, e.g., the late promoter and tripartite leader sequence. The resulting chimeric sequence may then be inserted into the adenovirus genome using in vitro or in vivo recombination. Insertion into a non-essential region of the viral genome (e.g., region E1 or E3), preferably, results in a recombinant virus capable of expressing a short RNA-binding protein of the present invention in infected hosts (See, e.g., Logan and Shenk (1984) Proc. Natl. Acad. Sci. USA 81:3655-3659). Specific initiation signals may also be required for efficient translation of inserted short RNA-binding protein-encoding nucleotide sequences. Non-limiting examples of such signals include the ATG initiation codon and adjacent sequences.

More particularly, when the sequence encoding the desired short RNA-binding protein, e.g., A-AC4 or S-AC4, comprises an initiation codon (and other necessary control sequences) and is inserted into an appropriate expression vector, no additional translational control signals may be needed. Of course, when such encoding sequences do not comprise such control sequences, certain exogenous transcriptional/translational control signals must be provided, including without limitation the ATG initiation codon, enhancer elements, transcription terminators, and/or others known in the art. Such exogenous translational control signals and initiation codons may be of a variety of origins, both native and non-native. Furthermore, those skilled in the art will appreciate that the initiation codon must be in phase with the reading frame of the desired short RNA-binding protein-encoding sequence to ensure transcription and translation of the entire insert.

In other embodiments, a host cell strain may be chosen that modulates the expression of the desired short RNA-binding protein, or modifies and processes the gene product in a specific fashion. Such modifications (e.g., glycosylation) and processing (e.g., cleavage) of protein products may be important for the function of the short RNA-binding protein. Different host cells have characteristic and specific mechanisms for the post-translational processing and modification of proteins and gene products. Appropriate cell lines or host systems may be selected to impart the desired modification and processing on the expressed short RNA-binding protein. Likewise, these modifications may occur without impacting the functionality of such protein and, therefore, are functionally, but not compositionally, the same.

In still other embodiments, as mentioned above (and shown in the examples below), the short RNA-binding proteins of the present invention may be expressed as fusion proteins, which may be readily purified using, for example, an antibody specific for the fusion protein. In another suitable example, a system described by Janknecht et al. allows for the efficient purification of non-denatured fusion proteins expressed in human cell lines (Janknecht, et al. (1991) Proc. Natl. Acad. Sci. USA 88:8972-8976). In such example, the nucleic acid sequence of interest, e.g., a sequence encoding A-AC4 (such as SEQ ID NO:3) or S-AC4 (SEQ ID NO:4), is subcloned into a vaccinia recombination plasmid, wherein the open reading frame of such polynucleotide is translationally fused to an amino-terminal tag consisting of six histidine residues. Extracts from cells infected with such recombinant vaccinia virus are loaded onto Ni²⁺ nitriloacetic acid-agarose column and histidine-tagged short RNA-binding proteins are selectively eluted with imidazole-containing buffers.

While the expression systems described above (and others) may be used to produce the short RNA-binding proteins contemplated by the present invention, in other embodiments, such proteins may be chemically synthesized. Such chemical synthesis may be conducted using any method known in art, such as either F-moc (9-fluorenylmethyloxycarbonyl) chemistry, essentially as described by D. Hudson (1988) or Boc (tert-butyloxycarbonoyl) chemistry. Techniques for amino acid synthesis are routinely employed by those of ordinary skill in the art using currently available laboratory equipment (e.g., Applied Biosystems, Inc., Foster City, Calif.).

Following chemical synthesis, peptides are generally purified using high pressure liquid chromatography (HPLC), and the integrity and authenticity of the peptides are determined by limited Edman degradation followed by traditional sequencing, such as mass-spectrophotometric analyses and NMR analysis of the intact peptide. In addition, the synthetically produced short RNA-binding proteins are, preferably, analyzed for biological activity using, for example, any of numerous in vitro assays known in the art—examples of which are described below.

In certain embodiments of the present invention, one or more of the short RNA-binding proteins described herein is provided directly to a system, e.g., any desired in vitro system. For example, the invention provides that purified and concentrated forms of such proteins (and/or the short RNA-binding compositions described below) may be provided to an in vitro system. The amount of short RNA-binding protein provided will depend on the type and nature of the in vitro system, which may range, for example, from 1-10 ng, 10-100 ng, 100 ng-1 μg, 1 μg-500 μg, or greater than 500 μg. The type or nature of the in vitro system used will depend, of course, on the investigator's research. More specifically, for example, if an investigator desires to modulate the effects of endogenous and/or exogenous miRNAs and/or siRNAs on host post-transcriptional gene silencing (PTGS) in a particular plant cell (or insect, mammalian, bacterial, nematode, or other system), concentrated and/or purified forms of one or more of the short RNA-binding proteins described herein, e.g., A-AC4 or S-AC4, may be directly provided to the system.

In cases where an investigator uses concentrated and/or purified forms of the short RNA-binding proteins for direct application to a system, the protein preferably comprises suitable buffers, stabilizers, and/or other appropriate solvents. Non-limiting examples of compositions which are known to stabilize proteins (a.k.a. “stabilizers”) include glycerol, reducing agents, hydrophobic additives, antibacterial agents, such as sodium azide, protease inhibitors, ethylenediaminetetraacetic acid (EDTA), and/or other compositions well-known in the art. If stored for long durations, the invention provides that such purified and/or concentrated forms of the short RNA-binding protein should be stored at colder temperatures, such as 4° C. or below, or even lyophilized (prior to reconstitution and use by an investigator). The purified and concentrated forms of the short RNA binding proteins described herein, such as A-AC4, S-AC4, or substantially similar sequences, constitute further embodiments of the present invention. Still further, the invention encompasses purified and concentrated forms of the short RNA-binding proteins reconstituted in suitable buffers, stabilizers, or other appropriate solvents—to provide short RNA-binding compositions with preferred shelf life. In such embodiments, “purified and concentrated” short RNA-binding proteins, as such terms are used herein, may be approximately 60% pure, or preferably at least 70% pure, or more preferably at least 80% pure, or still more preferably at least 90% pure.

In other embodiments, the short RNA-binding proteins described herein may be administered in vivo within the subject system, i.e., by expressing the desired short RNA-binding protein within the system for which short RNA-binding is desired. Similar to the methods described above (for production of the short RNA-binding proteins described herein), the invention provides that a system may be provided with an expression cassette which comprises (i) a promoter sequence, (ii) a nucleic acid sequence encoding at least one short RNA-binding protein described herein, such as SEQ ID NO:1, SEQ ID NO:2, or substantially similar sequences, and (iii) a termination sequence. In such embodiments, the system selected may be a plant cell, mammalian cell, insect cell, bacteria, fungi, yeast, or associated virus. Thus, if an investigator desires to modulate the effects of miRNA-guided inhibition of PTGS in a plant cell, for example, the investigator may transform the plant system with an expression cassette of the present invention that encodes A-AC4. Similarly, if an investigator desires to modulate the effects of double-stranded siRNA- and single-stranded miRNA-guided inhibition of PTGS in a plant cell, the investigator may transform the plant system with an expression cassette of the present invention that encodes S-AC4.

The methods of the present invention may further comprise a monitoring step. More specifically, the invention provides that the concentration of short RNA molecules within a system may, optionally, be monitored following the introduction of at least one short RNA-binding protein (or composition) described herein. The methods by which the concentration of short RNA molecules within a system may, optionally, be monitored include, without limitation, Northern blotting, Reverse Transcriptase PCR (RT-PCR), or any other techniques which allows for the detection and/or quantification of short RNA molecules. Such monitoring may be conducted after a system is directly provided with purified forms of a short RNA-binding protein, e.g., in an in vitro system, or after a system has been transformed with an expression cassette of the present invention and begins to express the short RNA-binding protein.

According to further embodiments of the present invention, kits are provided which may be used for binding short RNA molecules. In certain embodiments, for example, such kits comprise a purified and/or concentrated short RNA-binding protein described herein (or compositions which further include appropriate buffers, additives, reagents and stabilizers). In other related embodiments, the kits may comprise an expression cassette, which include a nucleic acid sequence encoding a short RNA-binding protein described herein, which may also include appropriate salts, buffers, enzymes, and other compositions for preferred shelf life.

The following examples are provided to illustrate further aspects of the present invention. These examples are illustrative only and are not intended to limit the scope of the invention in any way.

### EXAMPLES

### Example 1

### In vivo Binding of A-AC4 with Single-stranded Short RNA Molecules

This example demonstrates the use of A-AC4 to study the effects of miRNA-based inhibition on host mRNA (gene) expression in vivo. MicroRNAs play a major role in development by regulating host gene expression at the post-transcriptional level. Accordingly, the invention provides that, in certain embodiments, A-AC4 (or substantially similar sequences) may be used to investigate the importance or effects of miRNA-mediated inhibition of host mRNA expression, namely, by selectively binding (or otherwise inhibiting the effects of) such miRNAs in the host. By way of example, the following demonstrates the ability of A-AC4 to bind single-stranded short RNA molecules in vivo, such as miR159 (SEQ ID NO:5), which represents one of the most abundant miRNAs in leaf tissue. Still further, the effect of A-AC4 on MYB expression levels was measured, which is typically under negative regulation by miR159.

Gene Constructs and Transgenic Plants—Coding sequences of AC2 and AC4 genes from ACMV and EACMCV were PCR amplified and operably linked to the Cauliflower mosaic virus 35S promoter (CaMV 35S) and nopaline synthase (NOS) polyadenylation sequences in a binary vector, pBin, (which also contained a kanamycin resistance gene) (ACMV-AC2=SEQ ID NO:6; EACMCV-AC2=SEQ ID NO:7; ACMV-AC4=SEQ ID NO:2; and EACMCV-AC4=SEQ ID NO:8). The binary vector was subsequently introduced into an Agrobacterium tumefaciens strain GV3101. Transformation of Arabidopsis thaliana (ecotype Columbia plants) was performed using a floral dipping method (Clough S. J. and Bent, A. F. (1998) Floral dip: a simplified method for Agrobacterium-mediated transformation. Plant J. 6, 735-743). The test plants were transformed with the A. tumefaciens strain harboring the expression constructs described above, whereas the controls were transformed with an empty vector (the pBin vector without the AC2 and AC4 expression cassettes). Seeds from such transformants were selected for kanamycin resistance (50 mg/L).

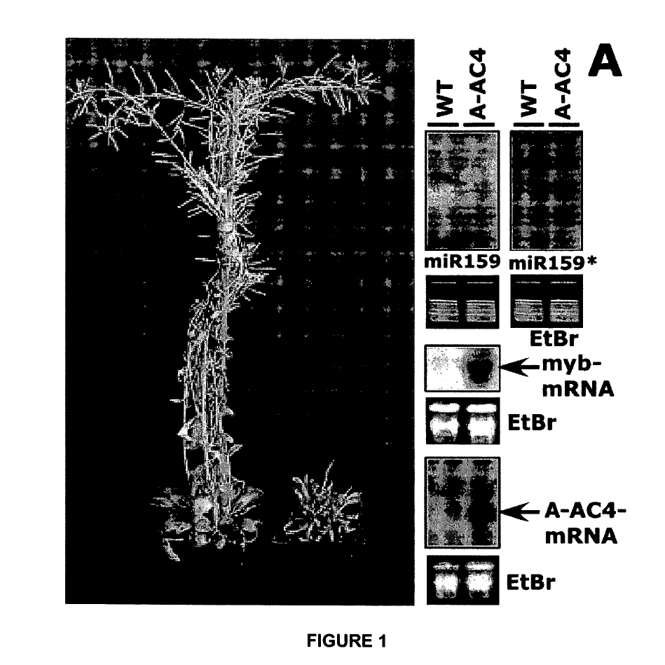

Northern blot analysis—Total RNA (20 μg) was isolated from a pool of leaf tissue collected from transgenic Arabidopsis using an RNA isolation kit. (Qiagen, Germantown, Md.). Hybridization, washes, and quantification of positive signals were conducted as previously described (Vanitharani, R., et al. (2003) PNAS 100, 9632-9636). The coding sequences of AC4 of ACMV (SEQ ID NO:3) and MYB (SEQ ID NO:9) were used for probes. The northern blot hybridization revealed a several fold increase of MYB-mRNA accumulation in A-AC4 transgenic Arabidopsis, relative to the level accumulated in vector-transformed control plants (“WT”) (FIG. 1).

The northern blotting results described above were confirmed by quantitative real-time RT-PCR (data not shown). More specifically, total RNA was isolated from wild type (“WT”) and AC4-transgenic tissue as described above, and cDNAs were synthesized from DNase (Invitrogen Corporation, Carlsbad, Calif.) treated RNA samples using MMLV Reverse Transcriptase (New England Biolabs, Beverly, Mass.) according to the manufacturer's instructions. Quantitative RT-PCR assays were performed using a Bio-Rad, Inc. (Hercules, Calif.) iCycler instrument, the cDNAs described above as template, MYB-gene specific primers, and SYBR green. As an internal control, the 5′-region of the ubiquitin gene was amplified using specific primers (Bovy, A., et al. (2002) Plant Cell 14, 2509-2526). As stated, the real-time RT-PCR results (not shown) supported the northern blot analysis described above.

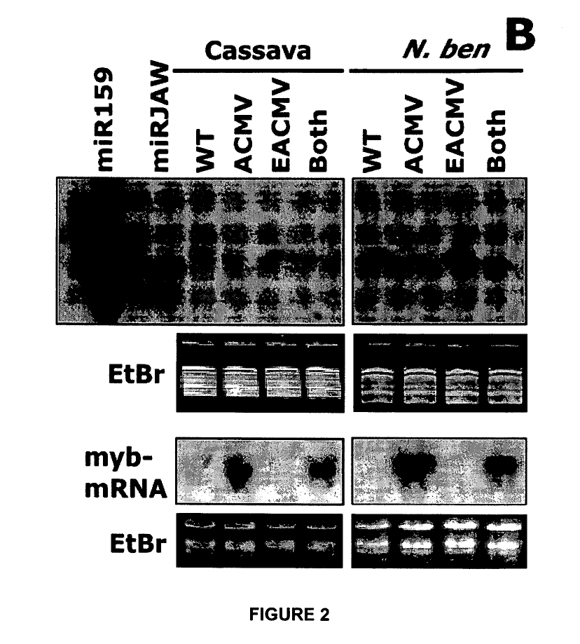

To further confirm that down-regulation (or inhibition) of miRNAs and up-regulation of target mRNAs by A-AC4 was not an artifact of the transgenic system, the levels of miRNA and target mRNA were measured by transiently expressing A-AC4 in N. benthamiana and cassava leaves by agro-infiltration (which was performed as described in Chellappan, P., et al. (2004) Journal of Virology 78, 7465-7477; and Chellappan, P., et al. (2004) Plant Molecular Biology 56, 601-611). Total RNA was extracted from infiltrated leaf-patches and northern blot analysis was carried out as described above. The northern blot hybridization revealed a several fold increase of MYB-mRNA accumulation in ACMV-infected N. benthamiana and cassava plants, relative to the level accumulated in control plants (FIG. 2)—an effect similar to the levels detected in the transgenic system described above and shown in FIG. 1. Here again, the data plainly show that A-AC4 inhibited the effects of miRNAs on MYB gene expression.

Among other reported suppressors that have been studied in detail, A-AC4 is unique in that it down-regulates the level (or inhibits the effect) of miRNAs consistently and, therefore, causes a similarly consistent up-regulation of target mRNAs because of the lack of miRNAs to control expression levels. Accordingly, the ACMV-AC4 is a powerful tool that may be used to study the importance or effects of miRNA-mediated inhibition of host mRNA expression (or the effects of other single-stranded, short RNA molecules). As shown in FIG. 1, for example, transgenic expression of A-AC4 in Arabidopsis resulted in stunted plants with severe developmental defects, such as narrow rosette leaves and lack of reproductive tissue growth.

The invention provides that A-AC4, or substantially similar proteins, may be used in other systems to bind short RNA molecules, such as miRNAs, affect mRNA expression levels, and study the resulting effects. Interestingly, N. benthamiana and cassava plants infected with a related East African cassava mosaic Cameroon virus (EACMCV) or Indian cassava mosaic virus (ICMV) caused only mild symptoms (not shown). Similarly, in ACMV-infected, but not in EACMCV-infected cassava plants, a reduction in miR159, miR165/166 and miR171 levels was observed (data not shown)—thus reinforcing the unique short RNA molecule binding capacity of the A-AC4 protein (and substantially similar sequences).

### Example 2

### In vitro Binding of A-AC4 with Short RNA molecules

In this example, the ability of the ACMV-AC4 (“A-AC4”) (SEQ ID NO:1), ACMV-AC2 (“A-AC2”) (SEQ ID NO:10), EACMCV-AC4 (“E-AC4”) (SEQ ID NO:l1), and EACMCV-AC2 (“E-AC2”) (SEQ ID NO:12) proteins to bind synthetic short RNA molecules (the sense and anti-sense strands of siRNAs, miR159, and miR159*) in vitro was measured by electrophoretic mobility shift assays. Specifically, purified viral proteins (AC2 and AC4 from ACMV and EACMCV) were incubated with synthetic oligo ribonucleotides miR159 (SEQ ID NO:5) and miR159* (SEQ ID NO:13) and anti-sense strands of siRNA (siGFP) (SEQ ID NO:14). In addition, the purified proteins were incubated with double-stranded RNA molecules, namely, miR159::miR159* and double-stranded siGFP complexes (positive annealing of such duplexes was confirmed through electrophoresis prior to use in binding assays).

Protein purification and in vitro binding assay—The AC4 and AC2 coding sequences were PCR amplified with specific primers integrated with BamHI and XhoI restriction sites using Pfx-polymerase (Invitrogen Corporation, Carlsbad, Calif.) and cloned into pET41a (Novagen, Madison, Wis.). The proteins were expressed in E. coli strain BL21, and purified using Ni-NTA resin (Qiagen, Germantown, Md.) following the recommendations of the manufacturer. The RNA oligonucleotides (a) miR159; (b) miR159*; (c) miR-lin4 (SEQ ID NO:15); (d) miR-lin4* (SEQ ID NO:16); (e) sense- (SEQ ID NO:17) and antisense-siRNA sequences of siGFP (SEQ ID NO:14); (g) double-stranded siGFP; and (h) miRNA-duplexes were 5′-labeled using [γ-32P]ATP and T4-polynucleotide kinase (New England Biolabs, Beverly, Mass.). Single-stranded short RNAs were annealed to their complementary strands to form the siGFP and miRNA duplexes, as previously described (Vanitharani, et al. 2003), and were confirmed by gel-electrophoresis for double-stranded siRNA and miRNA duplexes prior to labeling.

Labeled probe was purified using spin columns (Amersham Biosciences, Piscataway, N.J.), quantified by scintillation counting, and diluted to 10,000 cpm for each binding reaction with 500 ng of purified A-AC2, A-AC4, E-AC2 or E-AC4 protein. Binding buffer contained 20 mM Tris-Cl pH 8.0, 50 mM KCl, 5 mM MgCl₂, 25 mM NaCl, 2.5 mM dithiothreitol and 10% glycerol. Reactions were incubated at 220° C. for 15 minutes and the complexes were resolved through polyacrylamide (8%) gel-electrophoresis in 0.5×TBE buffer.

Detection of miRNAs×Low molecular mass RNA was isolated as previously described (Chellappan, et al., 2004). Oligonucleotide sequences complementary to miR159, miR165/66 (SEQ ID NO:18/19), and miR171(SEQ ID NO:20) (Invitrogen Corporation, Carlsbad, Calif.) were 5′-labeled using [γ-32P]ATP and T4 polynucleotide kinase (New England Biolabs, Beverly, Mass.)—which were separately used as probes. Hybridization and washes were conducted as previously described (Chellappan, et. al., 2004). Synthetic miR159 and miRJAW (SEQ ID NO:21) were used as molecular size markers (Dharmacon, Lafayette, Colo.).

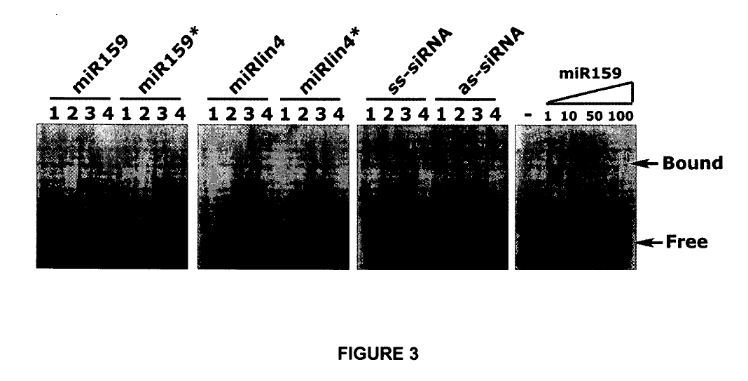

The results of the binding assays revealed (i) that A-AC4 binds to single-stranded miR159, miR159* (FIG. 3), (ii) that A-AC4 binds to single-stranded sense and anti-sense forms of siRNAs (FIG. 3), and (iii) that A-AC4 does not bind to duplex forms of miRNA159::miRNA159,* nor to double-stranded siRNAs (not shown). In contrast, the AC4 from EACMCV and the AC2 of ACMV and EACMCV did not show binding activity with single-stranded or duplex forms of both miRNAs and siRNAs (Data for E-AC2 is shown in FIG. 3).

The high-affinity binding of A-AC4 with single-stranded forms of miRNA, e.g., miR159, was further evidenced in a competition-binding assay when an excess of unlabelled competitor (miR159) was added with the labeled probes. In such competition assays, 10-, 50- and 100-fold excess (100, 500, and 1000 picomoles, respectively) of unlabelled miR159 oligonucleotides were added to the system. The gels were dried on Whatman paper, exposed to image screens and scanned using phosphorlmager (Amersham Biosciences, Piscataway, N.J.). As expected, the signal due to miRNA::AC4 protein complex formation decreased with an increasing amount of unlabelled miR159 as the competitor (FIG. 3).

In addition, the ability of purified A-AC4 protein to interact with miR-lin4, the first-identified miRNA which is involved in regulating developmental timing in Caenorhabditis elegans, was analyzed. Consistent with the results described above, A-AC4 protein bound to synthetic miR-lin4 and miR-lin4* (FIG. 3), but not to miR-lin4::miR-lin4* duplex molecules (not shown). Such results demonstrate the general miRNA-binding properties of A-AC4 (or, in other words, the single-stranded, short RNA molecule-binding properties of A-AC4).

### Example 3

### Isolation of AC4 Protein-miRNA Complex Using a Tethered 240 -O-methyl Oligonucleotide

To further confirm that A-AC4 is capable of binding single-stranded short RNA molecules, such as miRNAs, in vivo, an A-AC4::GFP fusion protein was expressed in Arabidopsis leaf-derived protoplasts. The A-AC4::miRNA complexes were assessed using an affinity binding assay and, more specifically, 2′-O-methly oligonucleotide as RNA bait—which has been successfully used to isolate RNA-associated proteins in Drosophila (Hutvagner, G., et al. (2004) PLoS Biology 2, 0465-0475).

In this example, the interaction of A-AC4 protein with miRNAs in vivo was determined by treating an extract from Arabidopsis protoplasts (which expressed the A-AC4::GFP fusion protein) with a 2′-O-methyl oligonucleotide complementary to miR159 (miR159*). The 2′-O-methyl oligonucleotide was tethered to streptavidin-coated magnetic beads via a 5′-biotin linkage. As a control, a 2′-O-methyl oligonucleotide complementary to the firefly (Photinus pyralis) luciferase (Pp-luc) sequence was used (SEQ ID NO:22). Arabidopsis thaliana (ecotype Columbia) leaf-derived protoplasts were transfected with a 35S::GFP alone or 35S::A-AC4::GFP fusion construct.

Immobilized 2′-O-methyl oligonucleotide affinity capture of AC4 —Synthetic biotinylated 2′-O-methyl oligonucleotides of (a) miR159* (complementary sequence of miR159) and (b) the antisense siRNA strand targeting the firefly (Photinus pyralis) luciferase (Pp-luc) mRNA (SEQ ID NO:23) were obtained from Integrated DNA Technologies, Inc. (Coralville, Iowa). The biotin group was attached to the oligonucleotides at the 5′-end via a six-carbon spacer arm.

The biotinylated 2′-O-methyl oligonucleotides, miR159* and unrelated Pp-luc (300 pmol), were incubated for 1 hour on ice in a binding buffer (40 mM Tris-Cl pH 7.4, 1 mM EDTA and 200 mM NaCl) with 50 μl of streptavidin coated Dynabeads M280 (Dynal, Brown Deer, Wis.)—to immobilize the oligonucleotides on the beads.

A-AC4 coding sequence was fused in-frame to the N-terminus of 35S::EGFP coding sequence by PCR using Pfx-polymerase. Protoplasts were inoculated with the 35S::AC4-EGFP fusion construct (or 35S::EGFP) using PEG-4000 and incubated at 280° C. without light. Cells were harvested 24 hours after transfection and homogenized in ice-cold buffer [(50 mM Tris-Cl pH 7.4, 100 mM KCl, 2.5 mM MgCl2, 0.1% (v/v) Triton X-100 and complete mini Protease Inhibitor cocktail (1 tablet/10 mL solution) (Roche, Basel, Switzerland)]. The extract was clarified by centrifugation at 12000×g for 10 minutes at 40° C. Extracts from protoplasts transfected with GFP-plasmid alone or A-AC4::GFP construct were incubated with immobilized 2′-O-methyl oligonucleotides (miR159* or Pp-luc) for 1 hour at 250° C. Next, the beads were collected using a magnetic stand (Dynal, Brown Deer, Wis.) and the unbound supernatant was analyzed for the depletion of miR159 on Northern blots.

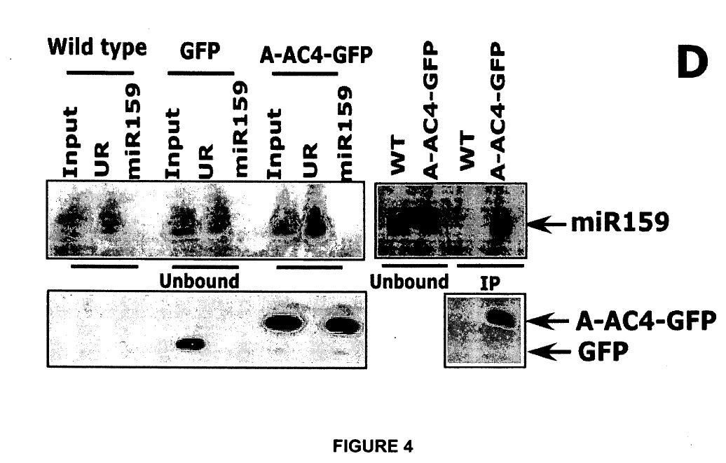

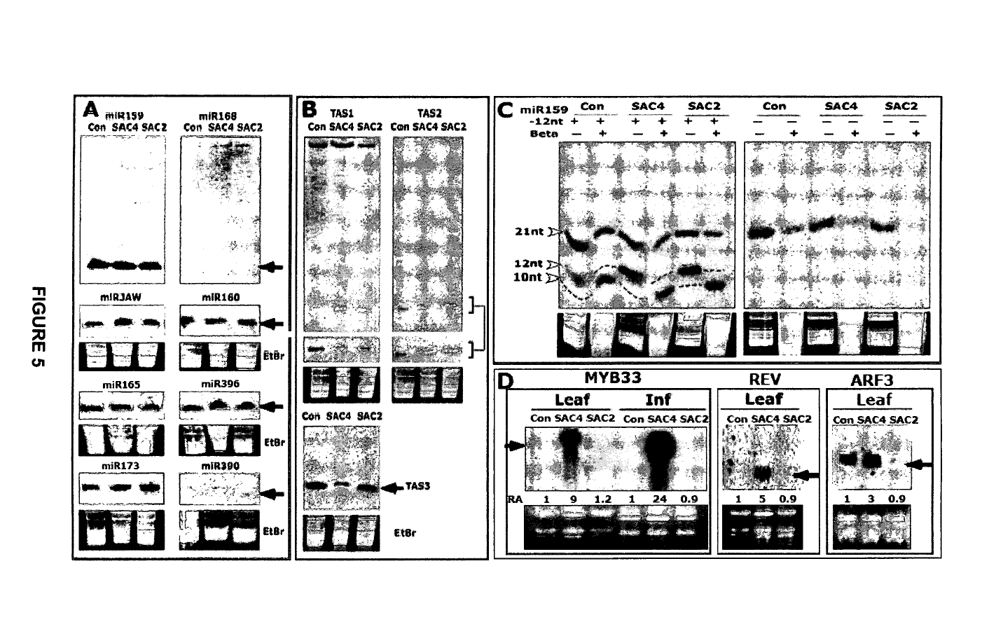

The ability of 2′-O-methyl-miR159* to retain miR159 was revealed by a reduction in the level of miR159 from the extracts as assayed in such Northern blots (FIG. 4). No reduction was observed in the samples treated with an unrelated 2′-O-methyl oligonucleotide complementary to the Pp-luc (“UR”) and in the input extracts (“Input”) (FIG. 4). As used herein, “Input” refers to the negative control. The results indicate that miR159-complementary 2′-O-methyl oligonucleotide efficiently binds to miR159 from the protoplast extract (FIG. 4).

The association of A-AC4 with miRNA, using miR159-complementary 2′-O-methyl oligonucleotide as RNA bait, was next analyzed. To isolate miR159-associated protein, extracts from protoplasts expressing GFP alone or A-AC4::GFP fusion protein were separately incubated with 2′-O-methyl miR159-complementary oligonucleotide immobilized to magnetic beads, as described above. After washing, the beads were boiled for 10 minutes in 20 μl of SDS loading buffer. Proteins were fractionated through 12% SDS-PAGE and transferred to nitrocellulose membrane (Bio-Rad, Hercules, Calif.). The transferred proteins were detected through Western blotting using anti-GFP monoclonal antibody (Clontech, Palo Alto, Calif.), as previously described (Chellappan, et al., 2004).

The GFP-tagged A-AC4-protein::miR159 complex copurified with the tethered 2′-O-methyl-miR159* oligonucleotide (FIG. 4, lane 9), but not with the unrelated oligonucleotide (FIG. 4, lane 8). These results demonstrate the association of A-AC4 protein with miRNAs in vivo. As shown, the GFP protein and A-AC4::GFP fusion proteins were detected in the input samples (FIG. 4; lane 4 and 7).

### Example 4

### Evaluation of AC4 Protein-miRNA Complex Through Immunoprecipitation

The association of A-AC4-protein::miRNA was further evaluated by immunoprecipitation using 35S::A-AC4::GFP fusion constructs in Arabidopsis protoplasts and anti-GFP monoclonal antibodies. More specifically, immunoprecipitation of GFP-tagged A-AC4 protein complexes was performed by treating total protoplast extract with 50 μg/mL Protein A-agarose beads (Roche, Basel, Switzerland) for 30 minutes at 40° C. The cleared extract was subsequently incubated with anti-GFP monoclonal antibody at a concentration of 1:2000 (Clontech, Palo Alto, Calif.) for 1 hour at 40° C. This incubation was followed by another Protein A-agarose (150 μg/mL) incubation for 3 hours at 40° C. The agarose beads were subsequently washed three times with ice-cold homogenization buffer.

RNA was eluted from the immune complexes by digestion with 1 mg/mL proteinase K (200 mM Tris-Cl (pH 7.4), 25 mM EDTA, 300 mM NaCl, 2% (w/v) SDS) at 50° C. for 30 minutes, followed by extraction with phenol-chloroform and recovered by ethanol precipitation. The recovered immunoprecipitated RNA was resuspended in 20 μl of formamide-containing loading buffer.

The recovered, immunoprecipitated RNAs (“IP”) were analyzed by Northern blotting. Specifically, the immunoprecipitated RNAs were detected using a 5′-labeled complementary sequence of miR159 probe (SEQ ID NO:13). miR159 was detected in the input samples (“Unbound”) and in the immuno complex obtained from protoplasts transfected with A-AC4::GFP fusion construct (“IP”/“A-AC4-GFP”) (FIG. 4, top, right blot), but not in the immuno complex obtained from control non-transfected protoplasts (“IP”/“WT”) (FIG. 4, top, right blot).

Presence of A-AC4::GFP fusion protein in the immune complexes was analyzed by Western blots using anti-GFP monoclonal antibody (within the input samples (not shown) and immunoprecipitated fractions). Western blot analysis of the input samples and immunoprecipitated fractions revealed the presence of A-AC4::GFP fusion protein in the immune complex obtained from A-AC4::GFP expression construct, but not in non-transfected control protoplasts (FIG. 4, lower, right blot).

### Example 5

### Binding of S-AC4 with Single- and Double-stranded Short RNA Molecules

This example demonstrates the use of S-AC4 to study the effects of miRNA- and siRNA-based inhibition on host mRNA (gene) expression.

Gene constructs and Transgenic Plants—In this example, the AC2 (S-AC2) and the AC4 (S-AC4) (e.g., SEQ ID NO:4) coding sequences from Sri-Lankan cassava mosaic virus (SLCMV) were PCR amplified using Platinum Pfx-DNA polymerase (Invitrogen Corporation, Carlsbad, Calif.), fused to the Cauliflower mosaic virus 35S promoter (CaMV) and NOS polyadenylation sequences in a pCAMBIA2300 binary vector. These vectors were subsequently introduced into an Agrobacterium tumefaciens strain GV3101. Arabidopsis thaliana ecotype Col-O plants were transformed using the A. tumefaciens strain harboring the S-AC2, S-AC4 expression constructs (or the empty vector for control) by the floral dipping method referenced above. Tranformants were selected for kanamycin resistance (50 mg/L). Transgenic plants were grown in standard green house conditions.

RNA isolation and blot analysis—For the studies involving analysis of Arabidopsis RNA levels, which are described in this example, low molecular mass RNA was extracted from young leaf tissues of S-AC4, S-AC2 and vector transformed control plants using TRIzol reagent (Sigma Chemical Co., St. Louis, Mo.). For detection of miRNAs and ta-siRNAs, gel blots containing 40 μg of the short RNA molecules (per lane) were hybridized with 5′-end labeled complementary DNA oligonucleotide (IDT) probes, which were prepared using γ32P-ATP and T4 polynucleotide kinase (NEB, lpswich, Mass.). Standard hybridization conditions and washes were employed.

For detection of mRNAs, 20 μg of total RNA was extracted using an RNA isolation kit from Qiagen, Inc. (Germantown, Md.), from young leaf and inflorescence tissues of S-AC4, S-AC2 and vector-transformed control plants. The extracted RNA was resolved on a 1.5% formaldehyde denaturing gel and blotted to HybondN+ membrane (Amersham Biosciences, Piscataway, N.J.). The coding regions of MYB33, REV and ARF3 were used for making gene-specific probes. Superscript RT-PCR kit (Invitrogen Corporation, Carlsbad, Calif.) was used for the first-strand cDNA synthesis from the Arabidopsis total RNA. Blot hybridization was carried out using gel-purified corresponding PCR fragments, labeled with α-32P-dCTP using a random-primer labeling kit (Stratagene, La Jolla, Calif.). To detect the transgene mRNAs in the S-AC4 and S-AC2 plants, the coding sequences of S-AC4 and S-AC2 were PCR-amplified, labeled with α-32P-dCTP and used as probes.

In-vitro binding assays—The RNA oligonucleotides used in this example were obtained from Dharmacon, Inc. (Chicago, Ill.) or Integrated DNA Technologies (IDT) (Coralville, Iowa). RNA oligonucleotides miR159 (SEQ ID NO:5), miR159* (SEQ ID NO:13), lin-4 (SEQ ID NO:15), lin-4* (SEQ ID NO:16), and precursor-miR159 (SEQ ID NO:40 and 41) were obtained from Dharmacon, Inc. miR165 (SEQ ID NO:18), miR165* (SEQ ID NO:33), miR34 (SEQ ID NO:34) and miR34* (SEQ ID NO:35), and the modified forms of miR165 and miR165* with a 2′-O-methyl group at the last nucleotide position, were obtained from Integrated DNA Technologies (IDT). The coding regions of the AC4 and AC2 genes were PCR amplified with specific primers integrated with BamHI and XhoI restriction sites using Platinum Pfx-polymerase, and cloned into BamHI and XhoI sites of pET41 a (Novagen, Madison, Wis.). The AC4 and AC2 proteins were expressed in E. coli strain BL21 and purified using Ni-NTA resin (Qiagen, Germantown, Md.) under native conditions.

Single-stranded short RNAs were annealed to their complementary strands—to form duplexes (i.e., double-stranded short RNA molecules)—and were confirmed by gel-electrophoresis prior to labeling with [γ-35P]ATP using T4-polynucleotide kinase. Labeled probe was purified using a nucleotide purification kit (Qiagen, Germantown, Md.), quantified by scintillation counting. For the binding assays described in this example, 20,000 cpm of such probe was used for each binding reaction with 500 ng of purified S-AC4-GST fusion protein. For the competition assays described herein, 10, 50 and 100-fold excess of unlabeled RNA-oligonucleotides were used. The gels were dried, exposed to image screens and scanned using Phosphorlmager (Molecular Dynamics).

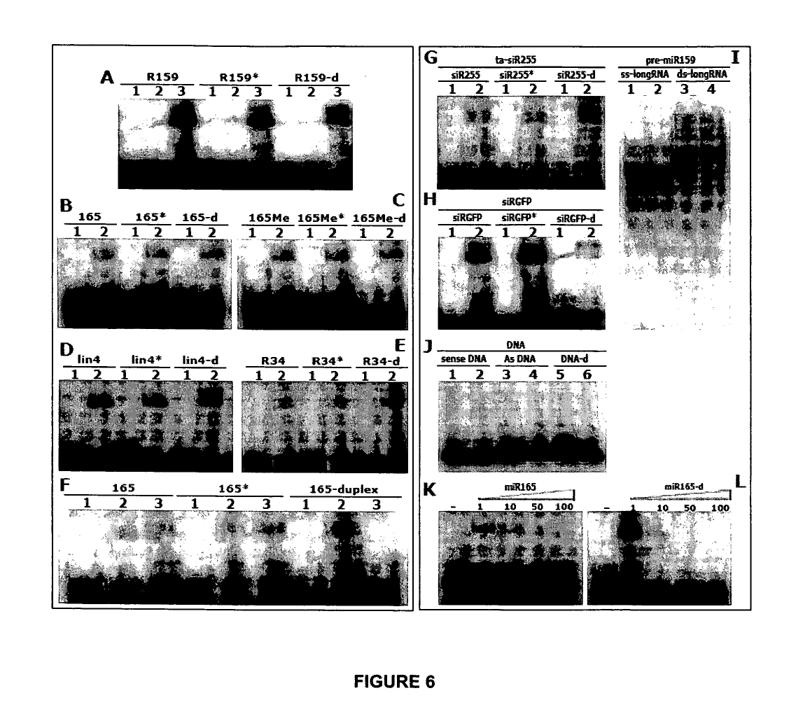

Evaluation of S-AC4 and its effect on the levels of mRNA targets of miRNA and ta-siRNA molecules—The effects of S-AC4 on the levels of target mRNAs that are regulated by miRNAs and ta-siRNAs were evaluated in Arabidopsis plants. Two miRNA targets were evaluated, namely, MYB33 and REV—which are normally under negative regulation by miR159 (SEQ ID NO:5) and miR165/166 (SEQ ID NO:18/19), respectively. MYB-domain transcription factors play a significant role in meristem, leaf and flower development. Leaf polarity is set by the refined expression of PHV, PHB and REV—all three of which are targets for a single miR165/166 short RNA molecule. Transgenic plants expressing S-AC4 showed severe developmental abnormalities, including leaf crinkling, upward and downward curling, and defective flowers. Northern blot analysis revealed that MYB33 mRNA accumulation was increased by 50-fold in the leaves—and was also increased in the influoresence tissue of S-AC4 plants compared to vector-transformed control plants (FIG. 5, Panel D). Similarly, REV mRNA, whose expression was below the level of detection in control and S-AC2 plants, had increased dramatically in S-AC4 plants (FIG. 5, Panel D). These results show that S-AC4 increased the target mRNA levels of the single-stranded miR159 and miR165/166 short RNA molecules by several fold in transgenic Arabidopsis plants.

The role of S-AC4 in the accumulation of target mRNAs that are regulated by ta-siRNAs was also evaluated. Specifically, ARF3 was evaluated, a transcription factor target for TAS3 species of ta-siRNA, which plays a major role in auxin homeostasis and root development. Similar to miRNA targets, ARF3 transcript level (mRNA level) was enhanced by several fold in S-AC4 plants compared to control (FIG. 5, Panel D). By contrast, expression of S-AC2 did not affect both miRNAs and ta-siRNAs target mRNA levels in such plants.

S-AC4 binds to both single- and double-stranded forms of short RNAs—In this example, the S-AC4 protein was expressed in E. coli, purified as a GST fusion protein and used for in vitro binding assays. miRNAs originating from various sources were tested, including plants, C. elegans and mammals. S-AC4 protein was found to bind miR159 and miR165/166, the two miRNAs that regulate MYB-family and class III HD-Zip (PHB, PHV and REV) transcription factors, respectively, in Arabidopsis. The S-AC4 protein was found to bind single- and double-stranded forms of miR159 and miR165/166 (FIG. 6, Panels A and B).

The ability of S-AC4 to bind animal derived miRNA sequences was examined using lin-4 (SEQ ID NO:15, an miRNA from C. elegans) and miR34 (SEQ ID NO:34, an miRNA from mammals). S-AC4 was found to bind single- and double-stranded forms of lin-4 and miR34 (FIG. 6, Panels D and E), indicating that S-AC4 binds short RNA molecules in a sequence non-specific manner. The differential binding nature of S-AC4 and A-AC4 was further demonstrated within the same Electrophoretic Mobility Shift Assay (EMSA) (FIG. 6, Panel F). The foregoing data show that the S-AC4 and A-AC4 proteins have the capacity to suppress RNA (mRNA) silencing, wherein A-AC4 may be used to selectively bind single-stranded short RNA molecules (e.g., miRNAs) and S-AC4 may be used to selectively bind single- and double-stranded short RNA molecules (e.g., miRNAs and siRNAs).

The high affinity binding nature of S-AC4 to single- and double-stranded forms of short RNA molecules was further tested using 10, 50 and 100-fold excess unlabelled RNA molecules, which corresponded to single- or double-stranded forms of miRNA165. The strength of the radioactive signal decreased with increasing amounts of unlabeled miR165 or miR165::miR165* (FIG. 6, Panels K and L). These results further demonstrate the high affinity association of S-AC4 protein with both forms of miRNAs (i.e., single- and double-stranded forms).

It has been shown that miRNAs in Arabidopsis plants are methylated—specifically at the 2′ position of the last ribonucleotide. In this example, whether S-AC4 is able to bind methylated forms of short RNAs was also examined, using synthetic miR165/166 and miR165/166* sequences with a 2′-O-methyl group attached at the ribonucleotide at the 3′ end. S-AC4 was found to bind the single- and double-stranded forms of modified miR165/166 as efficiently as to the unmodified miRNAs (FIG. 6, Panel C).

Next, the ability of S-AC4 to bind siRNAs (or, more specifically, trans acting siRNAs (ta-siRNAs)) was studied. In this example, synthetic siR255 ta-siRNAs (SEQ ID NO:29) were used—which belong to the TAS1 group of ta-siRNAs. S-AC4 was found to bind single- and double-stranded forms of such ta-siRNAs in a manner similar to miRNAs (FIG. 6, Panel G). The sequence non-specific binding nature of S-AC4 to short RNA molecules was further assessed by measuring its ability to bind to siRNAs targeted to GFP (siGFPs). In such case, S-AC4 was found to bind single- and double-stranded forms of such siGFPs, further demonstrating the sequence non-specific binding nature of the S-AC4 protein (FIG. 6, Panel H).

In addition, whether S-AC4 binds to DNA sequences of similar size was tested. More specifically, single- and double-stranded forms of DNA oligonucleotides, 21 nucleotides in length, with a sequence corresponding to miR165, was used in a binding assay with S-AC4. The results showed that S-AC4 was unable to bind to such DNA sequences (FIG. 6, Panel J). The results demonstrate that S-AC4 binds only to short RNA molecules in a sequence non-specific manner, but not to DNA sequences of a similar size.

Finally, the ability of S-AC4 to bind long RNA molecules was studied. Specifically, long RNAs representing the precursor sequence of miR159 were used. Two strands were obtained separately and annealed to produce duplex forms of the long-RNAs (pre-miR159) (SEQ ID NO:40 and 41). The binding assays revealed that S-AC4 does not bind to single- or double-stranded forms of such pre-miRNA (FIG. 6, Panel I). In summary, these results clearly demonstrate that S-AC4 has the ability to selectively bind single- and double-stranded forms of short RNA molecules present in plants, C. elegans, and mammals, but not to similar DNA sequences or long RNA molecules.

The many aspects and benefits of the invention are apparent from the detailed description, and thus, it is intended for the following claims to cover all such aspects and benefits of the invention which fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A method for inhibiting the effects of miRNAs on gene expression, which comprises providing to a system a protein which comprises an amino acid sequence of SEQ ID NO:1 (A-AC4).

2. The method according to claim 1, wherein said protein is immobilized to a solid or semi-solid substrate.

3. The method according to claim 1, wherein the protein is the A-AC4 protein (SEQ ID NO:1).

4. The method according to claim 3, wherein said protein is provided directly to the system.

5. The method according to claim 4, wherein the system is a mammalian cell, insect cell, bacteria, fungi, yeast, or virus.

6. The method according to claim 1, wherein said protein is provided to the system by introducing an expression cassette to the system, wherein said cassette comprises (i) a promoter sequence, (ii) a nucleic acid sequence encoding said protein, and (iii) a termination sequence.

7. A method for inhibiting the effects of miRNAs and siRNAs on gene expression, which comprises providing to a system a protein which comprises an amino acid sequence of SEQ ID NO:2 (S-AC4).

8. The method according to claim 7, wherein said protein is immobilized to a solid or semi-solid substrate.

9. The method according to claim 7, wherein the protein is the S-AC4 protein (SEQ ID NO:2).

10. The method according to claim 9, wherein said protein is provided directly to the system.

11. The method according to claim 10, wherein the system is a mammalian cell, insect cell, bacteria, fungi, yeast, or virus.

12. The method according to claim 7, wherein said protein is provided to the system by introducing an expression cassette to the system, wherein said cassette comprises (i) a promoter sequence, (ii) a nucleic acid sequence encoding said protein, and (iii) a termination sequence.

13. A kit for inhibiting the effects of miRNAs on gene expression, which comprises a purified and concentrated protein composition comprising an amino acid sequence of SEQ ID NO:1 (A-AC4).

14. The kit according to claim 13, wherein the purified and concentrated protein composition includes at least one buffer and stabilizer.

15. A kit for inhibiting the effects of miRNAs and siRNAs on gene expression, which comprises a purified and concentrated protein composition comprising an amino acid sequence of SEQ ID NO:2 (S-AC4).

16. The kit according to claim 15, wherein the purified and concentrated protein composition includes at least one buffer and stabilizer.

17. A kit for inhibiting the effects of short RNA molecules on gene expression, which comprises an expression cassette which includes a nucleic acid sequence encoding an amino acid sequence selected from the group consisting of SEQ ID NO:1, SEQ ID NO:2, and combinations thereof.

18. The kit according to claim 17, which comprises (i) a first expression cassette which includes a nucleic acid sequence encoding an amino acid sequence of SEQ ID NO:1; and (ii) a second expression cassette which includes a nucleic acid sequence encoding an amino acid sequence of SEQ ID NO:2.

19. The kit according to claim 18, wherein the first expression cassette comprises the nucleic acid sequence of SEQ ID NO:3 and the second expression cassette comprises the nucleic acid sequence of SEQ ID NO:4.

20. The kit according to claim 19, wherein the first and second expression cassettes are lyophilized or suspended in a suitable buffer.


SEQUENCE LISTING (41 sequences)

SEQ ID NO: 1 - protein, 140 aa, African cassava mosaic virus
MSFSHTQSVLYPKNTCCHSFKHSLSHQTLSSLKSVGSCIRMGNLTCMPSSNSRAKSRLRTIVSSIVYTRAVAPVSTPTFKVPNPAQMSSPIWIRTETPSNGDDFRSMDDLLEEVNNQRMMLTPKRLTAAVSQRLLMSLGN

SEQ ID NO: 2 - protein, 100 aa, Sri Lankan cassava mosaic virus
MGNLTSMLCFSSKGSSRSQTIGNSIVYTQAVAPVSTPTYRALSPAQTSSPTSTRMETPSTGVSFRSMEDLQEGDSRQPTTLTPQHLTQAVSQRLLESLRS

SEQ ID NO: 3 - DNA, 423 bp, African cassava mosaic virus
atgtctttctcacatacccaaagtgttctatacccaaagaacacctgctgtcattcattcaaacactctctctcccatcaaaccctaagttcattaaaatctgtagggagctgcatcagaatggggaacctcacttgcatgccctcatccaattcgagggcaaaatcacgattacgaacaatcgtctcttcgattgtgtacacccgagctgtagcacccgtttccaccccaacattcaaggtgccaaatccagctcagatgtcaagtcctatctggataaggacggagacaccgtcgaatggggacgatttcagatcgatggacgatctgctagaggaggtcaacaatcagcgaatgatgcttacgccaaagcgcttaacagcggcagtaagtcagaggctcttaatgtcattagggaattag

SEQ ID NO: 4 - DNA, 303 bp, Sri Lankan cassava mosaic virus
atggggaacctcacctccatgctctgcttcagttcgaagggaagctcacgatcacaaacaatcggcaattcgattgtgtacacccaagcagtagcaccagtttccaccccaacatacagagcgctaagtccagctcagacgtcaagtcctacatcgacaaggatggagacaccatcgactggggtgagtttcagatcgatggaagatctgcaagagggggacagcagacagccaacgacgcttacgccgcagcacttaacacaggcagtaagccagaggctcttagagtcattaaggagttag

SEQ ID NO: 5 - RNA, 21 nt, Arabidopsis sp.
uuuggauugaagggagcucua

SEQ ID NO: 6 - DNA, 408 bp, African cassava mosaic virus
atgcaatcttcatcaccctcacagaaccactctactcaggttccaatcaaagtcagtcacagacaattcaagaagcgagccatccggcgtaggagagtggatcttgtctgtggctgttcatattacctccatatcaactgctccaatcatggatttacgcacaggggaactcatcastgctcctcaagcaatgaatggcgggtatacctgggaaataaacaatcccctgtatttcacaataaccaagcaccaacaacgaccattcctgctgaaccaggacatcataacagtccaggttcrattcaatcmcaacctgaggaaggagctggggattcacaaatgttttctcaacttcaggttctggacgcccttaaagcctcagactggtcgtttcttaaggggtcttag

SEQ ID NO: 7 - DNA, 408 bp, African cassava mosaic virus
atgcgctcttcatctccctcacagagccactcttctcctccaccgatcaaagccaggcacaggcaagctaagctcagagcacctagacgtcgacgcatcgacctaacttgtggctgttccatctatcgcagcattaattgtcacaaccatggatttacgcacaggggaagacattggtgctcttcaatggaggagtggcgcctttatctgggagattccaaatcccctatatttcacaatcctcaaccacgacagcatgccgttcaacatgactcacgacataatagatgtccagattcggttcaactacaacctgaggcaagcactggggatgcacaagtgttttctcaacttccggatctggactcgtttacatcctcagacttggcgtttcttcagaacctttag

SEQ ID NO: 8 - DNA, 234 bp, African cassava mosaic virus
atggggtgcctcatctccatgttctcatccaattcgaaggcaagttccaatgtaccaaccccagattcttcgatctcatttccccatcccgatcaacacatttccatccgaacattcagggagctaaatcatcgtccgatgtcaaggcttacattgaaaagggaggggaatttcttgacgatggaattttccaagtcgatgccagaagtgccaggggggagggccagcatttag

SEQ ID NO: 9 - DNA, 2027 bp, Artificial
  Coding sequence for MYB gene
tcggatctctatccagctttggtatccagcgtctgacagttctggattttgttttttttttctttccttatttagggtgggagcttgatgtctaaattgttactgtcagtggtttaattagaaaagagatgagttacacgagcactgacagtgaccataatgagtcaccagctgctgatgataatggaagtgactgcagaagtagatgggatggtcatgctctcaagaaaggcccttggagttcagctgaagatgatattcttattgactatgtgaataagcatggtgagggtaactggaatgctgtgcagaaacacaccagcttgtttcgttgtggtaaaagctgtcgtctaagatgggctaatcatctgaggccaaatttgaagaaaggagcttttagtcaagaagaagaacagcttatcgttgaattgcatgccaagatgggtaatagatgggcacgtatggctgcacatttgcctggccgaacggataatgagataaagaattattggaacactcgtatcaagaggcgacaacgagctggtttgccactttatcctcctgagatgcatgttgaggcacttgagtggagtcaagagtatgccaagagtagagttatgggagaagatagaagacatcaagatttcttgcagctggggagttgtgaatctaatgtcttctttgatactcttaattttaccgacatggtacctggtacttttgatctagcagatatgactgcctacaaaaatatgggtaactgtgcaagttctcctcgatatgaaaacttcatgacaccaacaatcccctcctcgaagcgactttgggaatctgagttgttgtatcctgggtgtagcagtaccataaagcaagaattctcgtcgcctgaacaattccggaacacatctccacaaacgatttccaaaacttgcagcttctcagttccttgtgatgttgagcatcctctctatggaaaccgacattcacctgttatgattccagatagccatacccctacggatggcattgttccttattctaagcccttatatggggcagtgaagctggagctcccttcattccaatattcagaaacaacatttgaccagtggaagaaatcgtcatctcctccacactctgatctccttgatccctttgatacttacattcagtctccaccaccaccaacggggggagaagagtcagatttatattcaaattttgatactggtctgctcgatatgttgcttctggaagccaagatcagaaataatagtacaaagaacaatttgtacaggagctgcgcttcaactattccatcagctgatcttggccaggttactgtatcccaaactaaatccgaggagtttgacaattcccttaagagcttcttggttcattccgaaatgtccacacaaaatgcagatgaaactccaccaaggcagagagaaaaaaagcggaaacctctcttggatataacaaggcctgacgttttgcttgcatcgagctggcttgaccatggtttagggattgtgaaagagacaggtagcatgagcgacgcactcgcggttctccttggcgatgacataggaaatgactatatgaatatgagtgttggggcatcctcaggagttgggtcttgttcttggagcaacatgccacctgtctgtcaaatgacagaactaccctaacgagcccgggaaatcctccactccattggtggtgtggattggattagagccgttcagaatctgtcagttctcacttctcatggtcatggactagcaactgccaatgcaagaacaatatcttttctttgagccttttgatcacagccgatactttgttggcttctgtaccaataacccataatgacgattcatcaggaggatgaagtggttgattttgtagaactggcttctttaaggactatggtatacatacagacagttgatttgattcaatgttaaacagagaattgtttcttttaatatatttctctttggattaaaaaaaaaaaaaaaaaa

SEQ ID NO: 10 - protein, 135 aa, African cassava mosaic virus
  misc_feature (59)..(59): Xaa can be any naturally occurring amino acid
MQSSSPSQNHSTQVPIKVSHRQFKKRAIRRRRVDLVCGCSYYLHINCSNHGFTHRGTHXCSSSNEWRVYLGNKQSPVFHNNQAPTTTIPAEPGHHNSPGSIQSQPEEGAGDSQMFSQLQVLDALKASDWSFLKGS

SEQ ID NO: 11 - protein, 77 aa, African cassava mosaic virus
MGCLISMFSSNSKASSNVPTPDSSISFPHPDQHISIRTFRELNHRPMSRLTLKREGNFLTMEFSKSMPEVPGGRASI

SEQ ID NO: 12 - protein, 135 aa, African cassava mosaic virus
MRSSSPSQSHSSPPPIKARHRQAKLRAPRRRRIDLTCGCSIYRSINCHNHGFTHRGRHWCSSMEEWRLYLGDSKSPIFHNPQPRQHAVQHDSRHNRCPDSVQLQPEASTGDAQVFSQLPDLDSFTSSDLAFLQNL

SEQ ID NO: 13 - RNA, 21 nt, Arabidopsis thaliana
gagcuccuuaaaguucaaaca

SEQ ID NO: 14 - DNA, 21 bp, Aequorea victoria
gaugaacuucagggucagctt

SEQ ID NO: 15 - RNA, 21 nt, Arabidopsis thaliana
ucccugagaccucaaguguga

SEQ ID NO: 16 - RNA, 21 nt, Arabidopsis thaliana
acaccugggcucuccggguac

SEQ ID NO: 17 - DNA, 21 bp, Aequorea victoria
gcugacccugaaguucauctt

SEQ ID NO: 18 - RNA, 21 nt, Artificial
  Synthetic miRNA molecule
ucggaccaggcuucauccccc

SEQ ID NO: 19 - RNA, 21 nt, Artificial
  Synthetic miRNA molecule
ucggaccaggcuucauccccc

SEQ ID NO: 20 - RNA, 21 nt, Artificial
  Synthetic miRNA molecule
ugauugagccgcgccaauauc

SEQ ID NO: 21 - RNA, 20 nt, Arabidopsis sp.
uuggacugaagggagcuccc

SEQ ID NO: 22 - RNA, 21 nt, Artificial
  Synthetic Pp-luc sequence
cguacgcggaauacuucgaaa

SEQ ID NO: 23 - RNA, 21 nt, Photinus pyralis
cguacgcggaauacuucgaaa

SEQ ID NO: 24 - DNA, 21 bp, Artificial
  Synthetic miRNA molecule
tggcatacagggagccaggca

SEQ ID NO: 25 - DNA, 21 bp, Artificial
  Synthetic miRNA molecule
tccccgacctgcaccaagcga

SEQ ID NO: 26 - DNA, 22 bp, Artificial
  Synthetic miRNA molecule
gtgatttctctctgcaagcgaa

SEQ ID NO: 27 - DNA, 21 bp, Artificial
  Synthetic miRNA molecule
ggcgctatccctcctgagctt

SEQ ID NO: 28 - DNA, 21 bp, Artificial
  Synthetic miRNA molecule
cagttcaagaaagctgtggaa

SEQ ID NO: 29 - DNA, 21 bp, Artificial
  Synthetic siRNA molecule
tacgctatgttggacttagaa

SEQ ID NO: 30 - DNA, 21 bp, Artificial
  Synthetic siRNA molecule
aagtatcatcattcgcttgga

SEQ ID NO: 31 - DNA, 21 bp, Artificial
  Synthetic siRNA molecule
gggatagacaaggtaggagaa

SEQ ID NO: 32 - DNA, 21 bp, Artificial
  Synthetic siRNA molecule
ggtcaagaaaaggccttacaa

SEQ ID NO: 33 - RNA, 21 nt, Artificial
  Synthetic miRNA molecule
ggaauguugucuggaucuagg

SEQ ID NO: 34 - RNA, 22 nt, Artificial
  Synthetic miRNA molecule
aggcagugugguuagcugguug

SEQ ID NO: 35 - RNA, 22 nt, Artificial
  Synthetic miRNA molecule
acggcuaccuucacugccaccc

SEQ ID NO: 36 - DNA, 21 bp, Artificial
  Synthetic miRNA molecule
tcggaccaggcttcatccccc

SEQ ID NO: 37 - DNA, 21 bp, Artificial
  Synthetic miRNA molecule
gggggatgaagcctggtccga

SEQ ID NO: 38 - RNA, 21 nt, Artificial
  Synthetic siRNA molecule
augcgauacaaccugaaucuu

SEQ ID NO: 39 - RNA, 21 nt, Artificial
  Synthetic siRNA molecule
gagauucagguugcaucgcgc

SEQ ID NO: 40 - RNA, 95 nt, Artificial
  Synthetic miRNA precursor molecule
uaaaaaaggauuugguuauauggauugcauaucucaggagcuuuaacuugcccuguuaaugcuuuuacucuucuuuggcuugaagggagcucuac

SEQ ID NO: 41 - RNA, 89 nt, Artificial
  Synthetic miRNA precursor molecule
guagagcuccuuaaaguucaaacaugaguugagcaggguaaagaaaagcugcuaagcuauggaucccauaagcccuaauccuuguaaag